(19) United States
(12) Patent Application Publication
(10) Pub. No.: US 2023/0304101 A1
(43) Pub. Date: Sep. 28, 2023

(54) MOLECULAR BEACON BASED NANOPROBES FOR DIRECTLY DETECTING ORGAN-SPECIFIC MATASTASIS RELATED BIOMARKERS OF TUMOR CELLS IN PERIPHERAL BLOOD, PREPARATION METHOD AND APPLICATION

(71) Applicant: Wuhan University, Wuhan (CN)

(72) Inventors: Sixue CHENG, Wuhan (CN); Di HAN, Wuhan (CN)

(21) Appl. No.: 18/105,233

(22) Filed: Feb. 2, 2023

(30) Foreign Application Priority Data
Mar. 28, 2022 (CN) 202210314411.2

(51) Int. Cl. C12Q 1/6886

(52) U.S. Cl. CPC C12Q 1/6886; C12Q 1/6818

(57) ABSTRACT

The disclosure provides a molecular beacon nanoprobe for directly detecting organ-specific metastasis related genes of tumor cells in peripheral blood. The molecular beacon nanoprobe is a nanoparticle formed by self-assembly of a polymer material, a positively charged protein, a functional polypeptide, and a molecular beacon of organ-specific metastasis related genes of tumor cells.

### CROSS-REFERENCE TO RELATED APPLICATIONS

Pursuant to 35 U.S.C. § 119 and the Paris Convention Treaty, this application claims foreign priority to Chinese Patent Application No. 202210314411.2 filed Mar. 28, 2022, the contents of which, including any intervening amendments thereto, are incorporated herein by reference. Inquiries from the public to applicants or assignees concerning this document or the related applications should be directed to.

### BACKGROUND

The disclosure relates to the field of biomedicine, and more particularly to molecular beacon nanoprobes for directly detecting organ-specific metastasis related RNA biomarkers of tumor cells in peripheral blood and a preparation method and use thereof.

Most cancer-related deaths are caused by metastasis of primary tumors to distant organs such as lung, bone, liver, and brain. Tumor metastasis is a complex process. As early as 1889, Paget found that the tumor metastasis had obvious organ tendency, and put forward a “seed and soil” theory for the tumor metastasis. When metastasis of tumor cells occurs, the tumor cells called “seeds” are compatible with a specific organ microenvironment equivalent to “soil”. Recurrence or metastasis after chemotherapy is a major clinical challenge in the treatment of cancers. At present, methods for treatment of metastatic tumors have always been ineffective, and early prognostic/predictive methods for determining which organs are most prone to metastasis are lacking. Patients are basically asymptomatic in an early development stage of cancers, and disseminated cancer cells may take years to decades to develop into radiologically detectable metastatic lumps. However, most patients are not diagnosed by current radiological methods, and are diagnosed only in an autopsy. Therefore, it is very required to develop a method for early screening and diagnosis of patients and for real-time monitoring of organ-specific metastasis of primary tumors. On the one hand, dynamic monitoring of tumor metastasis can be carried out at any time, so that early detection and early intervention can be achieved. On the other hand, timely evaluation of a curative effect and detection of a drug resistance mechanism can be carried out, so that an effective treatment plan is adjusted and selected in time to achieve the purpose of accurate treatment of cancers, so as to improve the research, prevention, and control efficiency of cancer metastasis.

The heterogeneity of tumors is an important reason why all molecular information of the tumors cannot be obtained in a single biopsy, and also a basis for drug resistance of the tumors. Therefore, dynamic evaluation of molecular typing of the tumors is required. A liquid biopsy, as a non-invasive tumor detection method, can be used for continuously and dynamically evaluating circulating tumor cells (CTCs) escaping from primary tumors at an early stage, and cell-free tumor products (such as circulating tumor DNA (ctDNA), exosomes, and cell-free DNA (cf-DNA)). The CTCs can provide molecular information including DNA, RNA, and proteins at multiple levels, while other indicators can only provide abnormal information at a genetic level. It can be seen that the CTCs have a significant effect in early screening, efficacy evaluation, prediction of recurrence and metastasis, and the like.

Since the CTCs are relatively few in blood, it is particularly important to develop a technology for efficient detection of the CTCs. The detection of the CTCs in the blood mainly includes cell enrichment and isolation, and downstream characterization.

The enrichment and isolation of the CTCs mainly include two ways: 1): the CTCs are obtained by density gradient centrifugation or filtration and other ways based on unique physical properties (such as density and size) of the CTCs; and 2): the CTCs are obtained by immunomagnetic bead sorting, antibody specific binding, and other ways based on expression of specific cell surface proteins by the CTCs, and a most commonly used tumor cell surface marker includes an epithelial cell adhesion molecule (EpCAM), cytokeratin (CK), and the like, however, non-epithelial tumor cells are not likely to be captured by this method. At present, a commonly used CTCs detection system based on a physical method mainly includes: ISET, MetaCell, CellSieve, Parsortix, OncoQuick, VitaAssay, and the like; and a CTCs detection instrument based on marker-specific cell surface proteins mainly includes: CellSearch, MagSweeper, MACS, EPISPOT, CellCollector, and the like.

These detection methods based on the enrichment and the characterization in sequence have the following disadvantages. 1. During the enrichment, the CTCs may be damaged, or some surface-specific markers are lost. 2. Real-time dynamic monitoring of the CTCs cannot be achieved. 3. Enriched cells have high dependence on various staining methods, so that a cumbersome process is caused, and meanwhile, a false negative detection result is obtained when only some tumor cells are stained with antibodies due to the deletion of surface antigens or the heterogeneity of tumor cells. Therefore, the detection system needs to be optimized and studied, and it is particularly important to develop nanomaterials capable of detecting the deletion of the surface antigens or the heterogeneous tumor cells and then enriching the cells without damaging the CTCs. As most similar nanomaterials that have been reported have poor biocompatibility, interference will be caused to some target genes to be detected, and RNA cannot be well encapsulated or compounded to hide the nanomaterials from RNases and the immune system. Therefore, it is urgent to develop nanomaterials with great biocompatibility that can precisely monitor gene expressions in cells in real time without degradation and immunogenicity.

### SUMMARY

A first objective of the disclosure is to provide molecular beacon nanoprobes for directly detecting organ-specific metastasis related RNA biomarkers of tumor cells in peripheral blood. The molecular beacon nanoprobes can be efficiently targeted to circulating tumor cells in a blood environment to achieve the purpose of efficient detection of tumor metastasis sites.

A second objective of the disclosure is to provide a method for preparing the molecular beacon nanoprobes for directly detecting organ-specific metastasis related RNA biomarkers of tumor cells in peripheral blood. The method is simple in preparation process and easy to adjust.

A third objective of the disclosure is to provide use of the molecular beacon nanoprobes for directly detecting organ-specific metastasis related RNA biomarkers of tumor cells in peripheral blood.

A solution adopted by the disclosure to achieve the first objective is a molecular beacon nanoprobes for directly detecting organ-specific metastasis related RNA biomarkers of tumor cells in peripheral blood. The molecular beacon nanoprobes are nanoparticles formed by self-assembly of polymer material, positively charged protein, functional polypeptide, and molecular beacons of organ-specific metastasis related genes of tumor cells.

Preferably, the polymer material comprises any one of aptamer conjugated hyaluronic acid, peptide conjugated hyaluronic acid, aptamer conjugated carboxymethyl chitosan, peptide conjugated carboxymethyl chitosan, aptamer conjugated sodium alginate, peptide conjugated sodium alginate, aptamer conjugated heparin sodium, and peptide conjugated heparin sodium.

Preferably, an aptamer comprises at least one of AS1411 (anti-nucleolin aptamer), SYL3C (anti-epithelial cell adhesion molecule aptamer), MUC1-aptamer (anti-mucoprotein 1 aptamer), EGFR-aptamer CL4 (anti-epidermal growth factor receptor aptamer), and ICAM-1-aptamer (anti-intercellular adhesion molecule-1 aptamer); and a polypeptide comprises any one of a cell-penetrating peptide TAT, a CXCR4 (chemokine receptor) targeting peptide T22, a VCAM-1 (human vascular endothelial cell adhesion molecule 1) targeting peptide VHPKQ, and a fusion peptide formed by fusion of these peptides.

Preferably, the positively charged protein comprises any one of protamine, histone, and lysozyme.

Preferably, the molecular beacon of organ-specific metastasis related RNA biomarkers of tumor cells comprises any one of a tumor marker molecular beacon, a tumor brain metastasis marker molecular beacon, a tumor lung metastasis marker molecular beacon, a tumor bone metastasis marker molecular beacon, and a tumor liver metastasis marker molecular beacon. The tumor brain metastasis marker molecular beacon is at least one of molecular beacons for detecting Ki67, Notch, SPOCK1, and TWIST2 genes. The tumor lung metastasis marker molecular beacon is a molecular beacon for detecting a cathepsin (CTSC) gene. The tumor bone metastasis marker molecular beacon is at least one of molecular beacons for detecting Jagged1, interleukin 11 (IL-11), and CTGF genes. The tumor liver metastasis marker molecular beacon is a molecular beacon for detecting a claudin-2 gene.

The molecular beacon is labeled with a fluorescence group at the 5′ end and a fluorescence quenching group at the 3′ end.

Preferably, the functional polypeptide comprises any one of a KALA peptide, other penetrating peptides, a targeting peptide, and a fusion peptide.

Fluorescence emitted by the fluorescence group in the molecular beacon can be quenched by the quenching group. When the molecular beacon binds to a target cell, a stem loop of the molecular beacon is opened, and the fluorescence group gets far away from the quenching group, so that a fluorescence signal can be detected.

The molecular beacon nanoprobe of the disclosure comprises various specific molecular beacons labeled with different colors of fluorescence groups, and each molecular beacon is used for recognition of a specific target in the circulating tumor cells (CTCs). When no target occurs, the fluorescence groups get close to the quenching groups. As the fluorescence groups stay in a quenched state due to transfer of fluorescence resonance energy, fluorescence is not emitted by the probe at this time. When the aptamer on the surface of the nanoparticle binds to a protein overexpressed on the surface of the CTCs and then enters the CTCs through actively targeted endocytosis, the nanoparticle is disintegrated in the CTCs, and then the molecular beacons are released into the cytoplasm. When the molecular beacons and specific nucleic acid strands containing target sequences in the cytoplasm are hybridized, a conformational change is caused, and the fluorescence groups get far away from the quenching groups to emit bright fluorescence. As a result, different targets can be detected in a same reaction. When a sample contains one or more than one target, corresponding targets can be recognized based on fluorescence colors. Metastasis sites of tumor cells and changes of nucleic acid levels in the tumor cells can be monitored in real time based on different colors of fluorescence and the fluorescence intensity. Cells in the blood of cancer patients mainly comprise blood cells and CTCs. Since red blood cells and platelets have no endocytosis ability, the nanoprobe will not enter the red blood cells and the platelets. Since surfaces of white blood cells have no proteins that can specifically bind to the nanoprobe, the nanoprobe will not enter the white blood cells. Therefore, the nanoprobe can be used for efficiently and accurately detecting the CTCs in the blood and distinguishing different types of the CTCs based on different fluorescence emissions to identify the tumor metastasis sites.

Preferably, a nucleic acid capable of being detected by the molecular beacon nanoprobes comprises any one of miR-21, miR-221, CXCR4 mRNA, CTSC mRNA, Jagged1 mRNA, Ki67 mRNA, and EGFR mRNA.

A solution adopted by the disclosure to achieve the second objective is a method for preparing the molecular beacon nanoprobes for directly detecting organ-specific metastasis related RNA biomarkers of tumor cells in peripheral blood. The method comprises the following steps:

(1) adding a certain amount of the positively charged protein and the functional polypeptide to deionized water to prepare a solution A, adding a certain amount of a molecular beacon solution to deionized water to prepare a solution B, and adding the solution A dropwise to the solution B for uniform mixing; and

(2) adding the polymer material to the mixed solution obtained in step (1) for continuous and uniform mixing to obtain the molecular beacon nanoprobe.

A synthesis principle is as follows. The nanoprobes are synthesized by a self-assembly method. A molecular beacon@ protamine/functional polypeptide, molecular beacon @ histone/functional polypeptide or molecular beacon@ lysozyme/functional polypeptide nanoprobe is formed by electrostatic interaction of positively charged protamine sulfate (PS), histone or lysozyme, a functional polypeptide, and a negatively charged molecular beacon (MB). Then, a negatively charged polymer material is added to the molecular beacon@ protamine/functional polypeptide, molecular beacon@ histone/functional polypeptide or molecular beacon@ lysozyme/functional polypeptide nanoparticle having a positively charged surface to prepare a molecular beacon@ protamine/functional polypeptide, molecular beacon@ histone/functional polypeptide or molecular beacon@ lysozyme/functional polypeptide nanoprobe.

Preferably, when the selected polymer material comprises hyaluronic acid, carboxymethyl chitosan, sodium alginate, and heparin sodium, the polymer material is aptamer conjugated or peptide conjugated first according to step I or II.

I. The polymer material containing a carboxyl group is dissolved in a PBS solution (PH=6). A catalyst EDC/NHS is added for activation at room temperature. Then an aminated aptamer or polypeptide are added for a reaction at room temperature. A product obtained after the reaction is put in a dialysis bag for dialysis, and then freeze-dried to obtain a functionalized polymer material.

II. A carboxylated aptamer or polypeptide is dissolved in a PBS buffer solution. A catalyst EDC/NHS is added for activation at room temperature. Then the polymer material is added for a reaction at room temperature. A product obtained after the reaction is put in a dialysis bag for dialysis, and then freeze-dried to obtain a functionalized polymer material.

In steps I and II, after the catalyst EDC/NHS is added, a molar ratio of —COOH to EDC to NHS in the solution is 1:1.2:1.2, and a molar ratio of the polymer material to the aptamer or the polypeptide is 10:1.

When the polymer material comprises hyaluronic acid, sodium alginate, and heparin sodium, a functionalized polymer material is prepared by the method I. When the polymer material is carboxymethyl chitosan, a functionalized polymer material is prepared by the method II.

Preferably, in step (2), after the functionalized polymer material is added, a mass ratio of the positively charged protein to the functional polypeptide to the molecular beacon to the functionalized polymer material in the mixed solution is 30:(1-3):(1-2.5):(5-15), and the positively charged protein has a concentration of 1-3 μg/μL.

A solution adopted by the disclosure to achieve the third objective is the use of the molecular beacon nanoprobe for directly detecting organ-specific metastasis related RNA biomarkers of tumor cells in peripheral blood. The molecular beacon nanoprobe is used in the field of preparation of reagents for detecting tumors, tumor brain metastasis, tumor lung metastasis, tumor liver metastasis, and tumor bone metastasis.

The disclosure has the following advantages beneficial effects:

Since all materials in the molecular beacon nanoprobe of the disclosure are biocompatible materials, no toxic or side effects of the blood cells and the CTCs will be caused by direct incubation in the blood, and the detection of target RNA biomarkers in the tumor cells will not be interfered with. The molecular beacon nanoprobe of the disclosure has a particle size, potential, and surface morphology that can meet the requirements of entering cells, and also has great stability and biocompatibility.

The molecular beacon nanoprobe of the disclosure is prepared based on the principle of electrostatic interaction, and all processes are carried out in an aqueous phase. The preparation process is simple and efficient, and the nanoprobe can be synthesized in only half an hour. A polymer chain on the surface of the synthesized nanoparticle can be connected to different targeted aptamers and polypeptide molecules, so that the nanoprobe can efficiently reach tumor cell sites in a complex blood environment. The nanomaterial can make the molecular beacons encapsulated inside to prevent enzymolysis of the beacons in the blood and an immunogenic reaction. In addition, after entering the CTCs, the nanoprobe is disintegrated in the CTCs to efficiently release the molecular beacons into the cytoplasm, so that efficient binding of the beacons and targeted nucleic acid sequences is achieved, bright fluorescence is emitted, and the purpose of recognizing target cells is achieved.

According to the preparation method of the disclosure, the synthesis process is free of toxicity, simple, and fast, the whole process is carried out in an aqueous phase, and mass production can be achieved.

The molecular beacon nanoprobe of the disclosure can be directly incubated in the blood of cancer patients for real-time detection of different nucleic acid molecules in tumor cells in a living cell state. In this way, cell damage caused by enriching cells first is avoided. When a molecular beacon delivery system of the disclosure is used for detecting metastasis sites of the CTCs in the blood, the CTCs do not need to be enriched first, and the nanoparticle can be directly incubated in the blood for detecting different kinds of nucleic acid molecules in living cells with different colors of fluorescence in the entire circulatory system. The molecular beacon nanoprobe can be used for early detection of cancers, identification of metastasis sites of cancers, and medication guidance, so as to improve the research, prevention, and control efficiency of cancer metastasis. Especially, the molecular beacon nanoprobe can be used for detection of early tumors, tumor brain metastasis, tumor lung metastasis, tumor liver metastasis, and tumor bone metastasis.

According to the molecular beacon nanoprobe of the disclosure, since a functional polypeptide is added, specific cell targeting, cellular uptake, and endosomal escape can be promoted. Therefore, the functional polypeptide is introduced to the core and/or surface of the nanoprobe to more effectively improve the delivery efficiency of the molecular beacons.

### BRIEF DESCRIPTION OF THE DRAWINGS

FIG. 1 is a diagram showing synthesis and an action principle of a molecular beacon nanoprobe of the disclosure;

FIG. 2 is a diagram showing an action principle of MB1/MB2/MB3@KALA/PS/SHA/IHA in Example 1 of the disclosure;

FIG. 3 shows synthesis processes of SYL-3C and ICAM-1-aptamer conjugated sodium hyaluronate materials in Example 1 of the disclosure;

FIG. 4 is a characterization diagram showing the morphology and particle size of an MB1/MB2/MB3@KALA/PS/SHA/IHA nanoparticle under a transmission electron microscope in Example 2 of the disclosure;

FIG. 5 is a diagram showing the fluorescence of MB1/MB2/MB3 incubated with two different nucleic acid molecules in a buffer solution for 2 hours in Example 2 of the disclosure;

FIG. 6 is a diagram showing fluorescence changes of free MB1/MB2/MB3 and an MB1/MB2/MB3@KALA/PS/SHA/IHA nanoparticle after standing in a buffer for different times under the presence of DNAase I without targeted nucleic acids in Example 2 of the disclosure;

FIG. 7 is a diagram showing the viability of breast normal cells MCF-10A, breast cancer cells MCF-7, and breast cancer cells MDA-MB-231 after co-incubation with MB1/MB2/MB3, IHA/SHA/PS/KALA, and an MB1/MB2/MB3@KALA/PS/SHA/IHA nanoparticle for 4 hours and 24 hours in Example 2 of the disclosure;

FIG. 8 is a diagram showing changes of targeted RNA (CTSC mRNA, CXCA4 mRNA, and Jag1 mRNA) in MCF-7 and MDA-MB-231 cells treated with an empty vector IHA/SHA/PS/KALA for 4 hours and MCF-7 and MDA-MB-231 cells without treatment with an empty vector in Example 2 of the disclosure;

FIG. 9 is a diagram showing the endocytosis of an YOYO-1-Hairpin@KALA/PS/SHA/IHA nanoparticle in MCF-7 and MDA-MB-231 cells in Example 3 of the disclosure;

FIG. 10 is a diagram showing expression levels of genes in cells (low metastatic breast cancer cells MCF-7, and highly metastatic breast cancer cells MDA-MB-231) with different metastasis abilities after detection with an MB1/MB2/MB3@KALA/PS/SHA/IHA nanoparticle in a simulated blood environment in Example 3 of the disclosure;

FIG. 11 is a diagram showing expression levels of different genes in CTCs after detection with an MB1/MB2/MB3@KALA/PS/SHA/IHA nanoparticle in a real patient blood environment in Example 3 of the disclosure; and

FIG. 12 is a diagram showing the endocytosis of an YOYO-1-Hairpin@KALA/PS/SHA/IHA nanoparticle and an YOYO-1-Hairpin@PS/SHA/IHA nanoparticle in MCF-7 and MDA-MB-231 cells.

### DETAILED DESCRIPTION

To better understand the disclosure, the following embodiments are to further illustrate the disclosure, but not used to limit the content of the disclosure.

Unless otherwise specified, technical means used in the examples are conventional means well known to those skilled in the art, and raw materials used are all commercially available commodities.

### Example 1 Synthesis and Use of a Molecular Beacon Nanoprobe Nanoparticle

1. Synthesis of a Molecular Beacon Nanoprobe

Specific processes are as follows. Sodium hyaluronate, sodium alginate, or heparin sodium (150 μg) dissolved in a PBS buffer solution (pH=6.0, 1 mL) was activated with a catalyst EDC/NHS (a molar ratio of —COOH to EDC to HoBt was 1:1.2:1.2) at room temperature for 1 hour. Then an aminated aptamer or polypeptide (150 μg) was added for a reaction at room temperature for 24 hours. Alternatively, a carboxylated aptamer or polypeptide (150 μg) was activated with a catalyst EDC/NHS at room temperature for 1 hour, and then carboxymethyl chitosan (150 μg) was added for a reaction at room temperature for 24 hours. A product obtained after the reaction of the four polymer materials was put in a dialysis bag (MWCO 10,000) for dialysis with ultrapure water for 3 days, and then freeze-dried to finally obtain a functionalized polymer material.

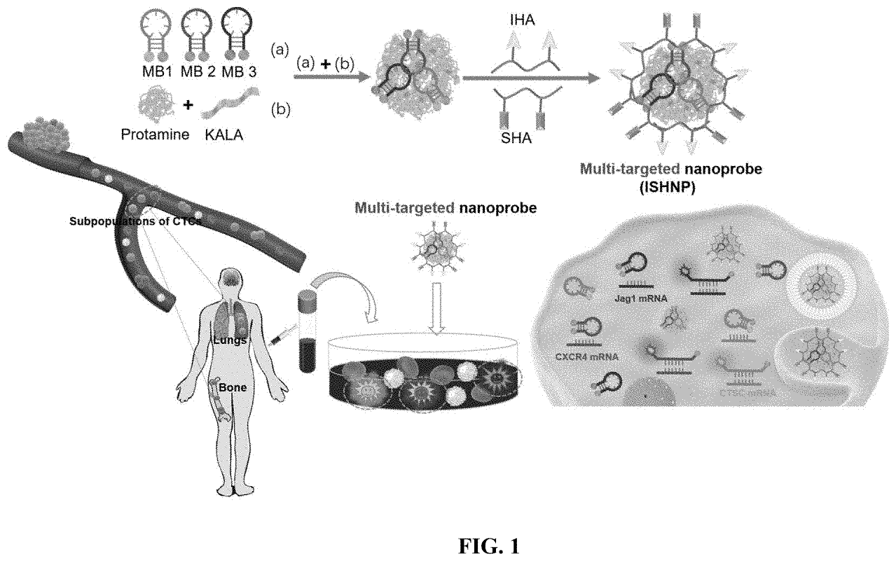

All reagents used for preparing the nanoparticle were dissolved in deionized water to obtain solutions with specific concentrations. A positively charged protein solution (1 μg/μL, 30 μL), a KALA solution (0.5 μg/μL, 6 μL), and deionized water (14 μL) were mixed to obtain a solution A with a total volume of 50 μL. A molecular beacon solution (210 nM, 21 μL) and deionized water (15 μL) were mixed to obtain a solution B with a total volume of 36 μL. The solution A was added dropwise to the solution B for slight mixing for 10 minutes. Then, the functionalized polymer material (1 μg/μL, 14 μL) was added for continuous mixing for 10 minutes to finally obtain a functionalized biopolymer material/positively charged protein/KALA/molecular beacon nanoparticle. A synthesis process and an action principle of the nanoparticle are shown in FIG. 1.

2. Synthesis of an MB1/MB2/MB3@KALA/PS/SHA/IHA (ISHNP) Nanoparticle

An example is given to further illustrate the synthesis process and experimental effect of the disclosure. According to investigation and study,

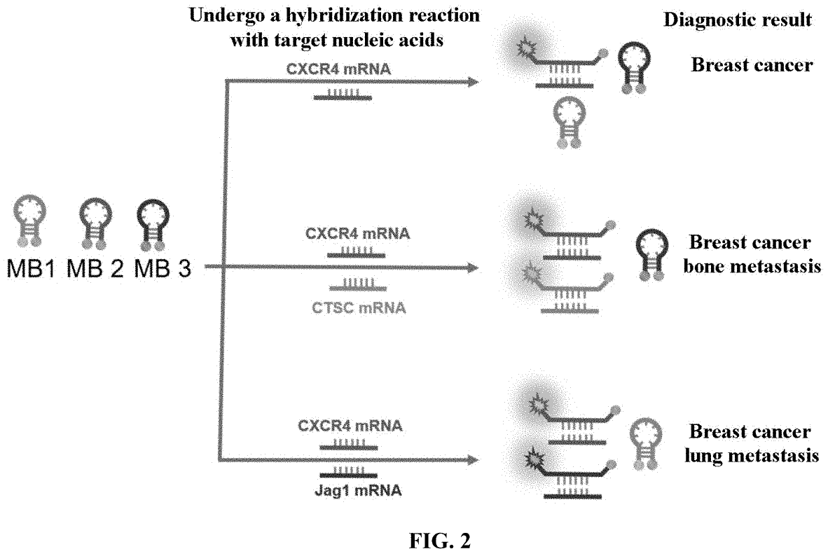

CXC chemokine receptor 4 (CXCR4) mRNA is overexpressed in almost all tumor cells, CTSC mRNA is overexpressed in breast cancer lung metastasis CTCs, and Jag1 mRNA (Jagged1 (Jag 1): a ligand of a transmembrane receptor protein Notch) is overexpressed in breast cancer bone metastasis CTCs. Therefore, in order to detect metastasis sites of early-stage cancer patients and metastatic cancer patients, the nanoparticle was used for encapsulating molecular beacons MB1/MB2/MB3. In addition, in order to make the nanoparticle better targeted to tumor cells, a polymer (sodium hyaluronate, HA) composing the nanoparticle was modified with an epithelial cell adhesion factor aptamer (SYL-3C) targeted to overexpression of cancer cells and an intercellular adhesion factor aptamer (ICAM-1 aptamer) to finally constitute an SYL-3C functionalized hyaluronic acid (targeted to epithelial tumor cells) and ICAM-1 aptamer functionalized hyaluronic acid (targeted to mesenchymal tumor cells)/positively charged protein/KALA/MB1/MB2/MB3 (MB1/MB2/MB3@KALA/PS/SHA/IHA, ISHNP) nanoparticle. A principle of the nanoparticle is shown in FIG. 2.

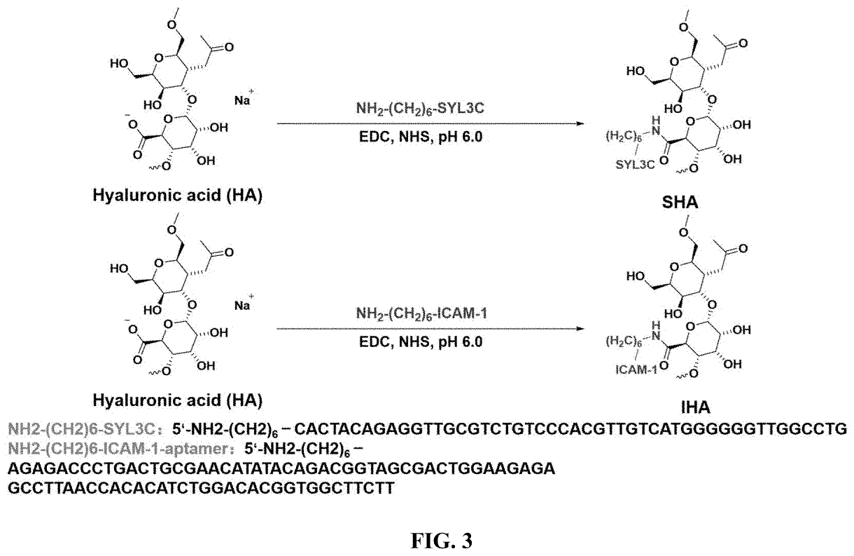

Synthesis processes of SYL-3C functionalized sodium hyaluronate and ICAM-1-aptamer functionalized sodium hyaluronate are shown in FIG. 3. The process of preparing a molecular beacon nanoprobe nanoparticle is as follows. (1) The sodium hyaluronate (150 μg) dissolved in a PBS buffer solution (pH=6.0, 1 mL) were activated with a catalyst EDC/NHS (a molar ratio of —COOH to EDC to HoBt was 1:1.2:1.2) at room temperature for 1 hour. Then an SYL-3C aptamer (150 μg) and an ICAM-1 aptamer (150 μg) were separately added for reactions at room temperature for 24 hours. Products obtained after the reactions were put in a dialysis bag (MWCO 10,000) for dialysis with ultrapure water for 3 days, and then freeze-dried to obtain SYL-3C functionalized hyaluronic acid and ICAM-1-aptamer functionalized hyaluronic acid respectively.

(2) All reagents used for preparing the nanoparticle were dissolved in deionized water to obtain solutions with specific concentrations. A positively charged protein solution (1 μg/μL, 30 μL), a KALA solution (0.5 μg/μL, 6 μL), and deionized water (14 μL) were mixed to obtain a solution A with a total volume of 50 μL. A molecular beacon solution MB1/MB2/MB3 (70 nM/70 nM/70 nM, 21 μL) and deionized water (15 μL) were mixed to obtain a solution B with a total volume of 36 μL. The solution A was added dropwise to the solution B for slight mixing for 10 minutes. Then, the SYL-3C functionalized hyaluronic acid (1 μg/μL, 7 μL) and the ICAM-1-aptamer functionalized hyaluronic acid (1 μg/μL, 7 μL) were added for continuous mixing for 10 minutes to finally obtain an MB1/MB2/MB3@KALA/PS/SHA/IHA nanoparticle.

The MB1/MB2/MB3@KALA/PS/SHA/IHA nanoparticle used in Examples 2 and 3 is prepared in this example.

### Example 2 Characterization of the MB1/MB2/MB3@KALA/PS/SHA/IHA Nanoparticle

1. Determination of the Particle Size, Potential, and Encapsulation Rate of the MB1/MB2/MB3@KALA/PS/SHA/IHA Nanoparticle

A specific implementation method is as follows. An MB1/MB2/MB3@KALA/PS/SHA/IHA nanoparticle solution prepared in Example 1 was diluted with deionized water to a total volume of 1 mL. The size and potential of the MB1/MB2/MB3@KALA/PS/SHA/IHA nanoparticle in the deionized water were measured by Zetasizer (Nano ZS, Malvern Instruments). Based on three independent tests, data were expressed as mean±standard deviation (SD). In order to determine the encapsulation rate of molecular beacons, the solution containing the MB1/MB2/MB3@KALA/PS/SHA/IHA nanoparticle was centrifuged at a specific rotation speed (10,000 rpm) at 4° C. for 1 hour, and then the amount of residual unprecipitated free molecular beacons in a supernatant was measured. The encapsulation rate of the molecular beacons was calculated as the amount of precipitated beacons to the total feeding amount. Experimental results are shown in Table 1. From the data in the table, it can be seen that the MB1/MB2/MB3@KALA/PS/SHA/IHA nanoparticle has a hydrodynamic size of less than 300 nm, and is suitable for cellular uptake. In addition, all the molecular beacons have an encapsulation rate of 90% or more.

TABLE 1

| | | | Encapsulation |
|---|---|---|---|
| | | | rate of |
| | | Potential | molecular |
| Name of sample | Size (nm) | (mV) | beacons (%) |
| molecular beacon@KALA/ | 140 +/− 3 | 17 +/− 3 | 90 +/− 1 |
| Positively charged protein | | | |
| molecular beacon@KALA/ | 200 +/− 9 | 11 +/− 1 | 92 +/− 1 |
| Positively charged protein/ | | | |
| Polymer material | | | |
| MB1/MB2/MB3@KALA/ | 275 +/− 14 | 16 +/− 1 | 94 +/− 1 |
| PS/SHA/IHA | | | |

2. Morphology of the MB1/MB2/MB3@KALA/PS/SHA/IHA Nanoparticle Under a Transmission Electron Microscope (TEM)

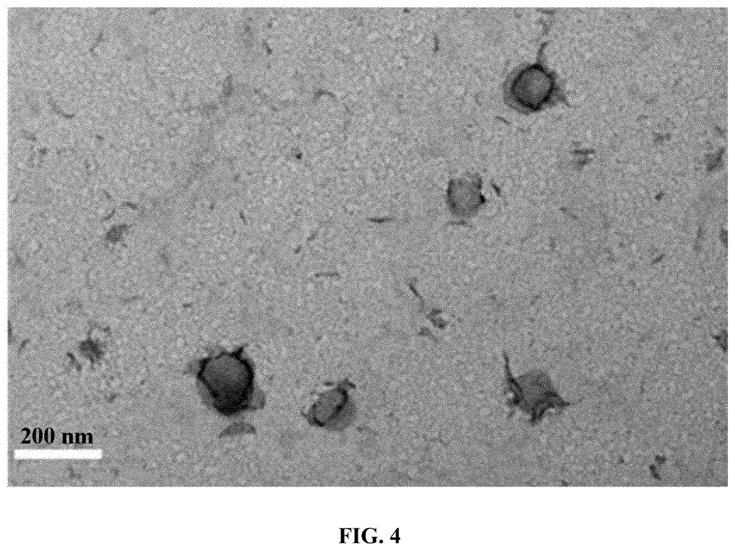

A specific implementation method is as follows. An ultra-thin carbon support membrane was infiltrated into a sample solution. After the MB1/I/MB2/MB3@KALA/PS/SHA/IHA nanoparticle was precipitated, a small amount of a phosphotungstic acid solution (0.001 mol/L) was added for negative staining by infiltration, and the solution was evaporated and air-dried at room temperature. At last, a sample was observed by a transmission electron microscope (JEM-2100). Experimental results are shown in FIG. 4. It can be seen from the figure that the MB1/1MB2/MB3@KALA/PS/SHA/IHA nanoparticle is in a uniformly dispersed spherical shape.

3. Specificity of MB1, MB2 and MB3

A specific implementation method is as follows. The MB1, MB2 and MB3 and nucleic acid molecules (comprising complementary targets CTSC mRNA, CXCR4 mRNA, Jag1 mRNA, and mismatched mRNA) were co-incubated in a 1×TNa buffer (pH=7.5, 20 mM). After co-incubation for 2 hours, fluorescence was measured by using a fluorospectrophotometer (RF-5301PC, Japan).

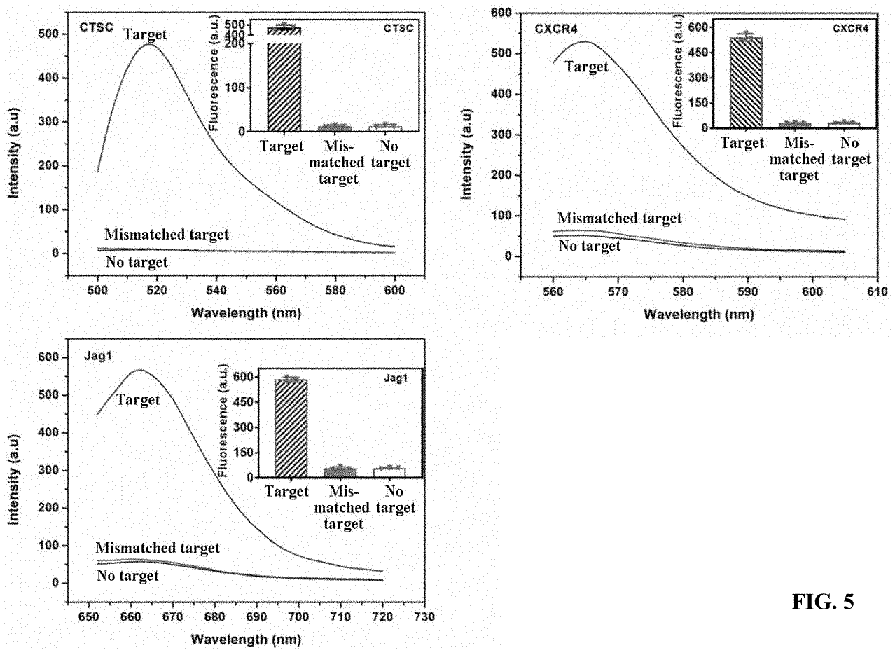

All experimental steps were repeated at least three times. Data was given as mean±standard deviation (SD). Experimental results are shown in FIG. 5. It can be seen from the figure that when MB1/MB2/MB3 is separately bound to the complementary targets CTSC mRNA, CXCR4 mRNA, and Jag1 mRNA, strong fluorescence is emitted. However, when the MB1, MB2 and MB3 is bound to the mismatched mRNA, respectively, almost no fluorescence is emitted. It indicates that the MB1, MB2 and MB3 beacons have great specificity, and according to the results shown, false positives in cells are avoided to a great extent.

4. Stability of an MB1/MB2/MB3@KALA/PS/SHA/IHA (ISHNP) Nanoparticle

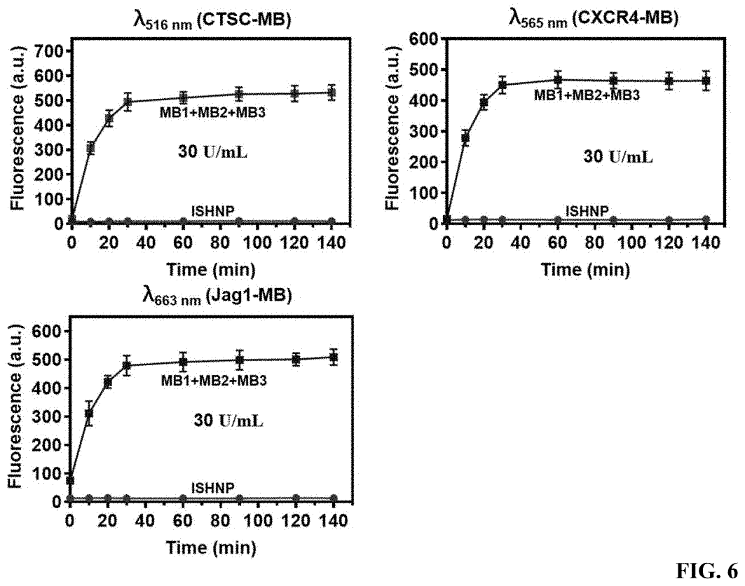

A specific implementation method is as follows. 30 U of DNase I was separately added to 1 mL of a 1×TNa buffer (pH=7.5, 20 mM) containing free MB1/MB2/MB3 and 1 mL of a 1×TNa buffer (pH=7.5, 20 mM) containing the MB1/MB2/MB3@KALA/PS/SHA/IHA nanoparticle. Standing was performed for 2 hours. Then fluorescence was detected by using a fluorospectrophotometer (RF-5301 PC, Japan). Experimental results are shown in FIG. 6. It can be seen from the figure that the MB1/MB2/MB3@KALA/PS/SHA/IHA nanoparticle has high stability, and can prevent a false positive signal caused by enzymolysis of the free MB1/MB2/MB3 encapsulated inside in cells.

5. Biocompatibility of the MB1/MB2/MB3@KALA/PS/SHA/IHA Nanoparticle

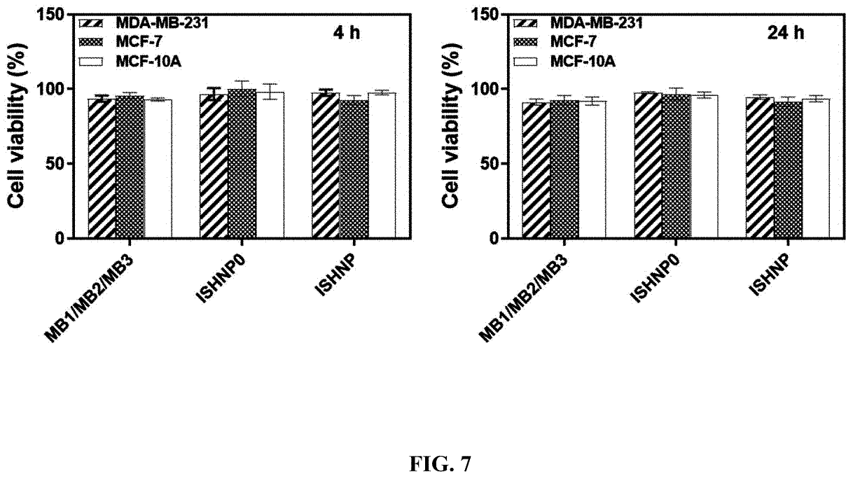

A specific implementation method is as follows. The in vitro toxicity of free MB1/MB2/MB3, an IHA/SHA/PS/KALA (ISHNP0) empty vector, and the MB1/MB2/MB3@KALA/PS/SHA/IHA (ISHNP) nanoparticle to tumor cells was determined by using a CCK-8 assay. 10⁴ MCF-10A, MCF-7, and MDA-MB-231 cells contained in a culture medium were separately inoculated into 6 96-well plates and cultured for 24 hours, and then 200 μL of the nanoparticle was inoculated into the 96-well plates. After incubation at 37° C. for four hours and 24 hours, 10 μL of CCK-8 was added to each well. At last, the absorbance at 480 nm was measured by using a microplate reader. Experimental results are shown in FIG. 7. It can be seen from the figure that all the free MB1/MB2/MB3, the IHA/SHA/PS/KALA (ISHNP0) empty vector, and the MB1/MB2/MB3@KALA/PS/SHA/IHA (ISHNP) nanoparticle have a cell viability of 90% or more, indicating that the material has great biocompatibility.

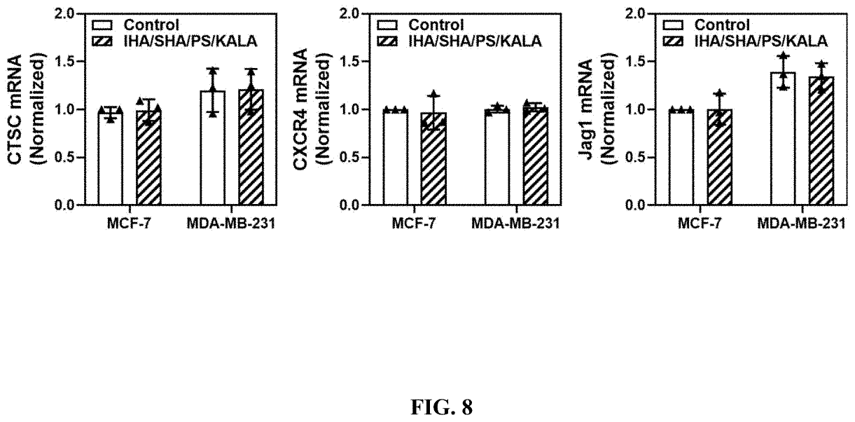

The interference of an empty vector IHA/SHA/PS/KALA nanoparticle on targets in tumor cells was determined by using a PCR test. 10⁶ MCF-7 and MDA-MB-231 cells contained in a culture medium were separately inoculated into 6-well plates and cultured for 24 hours, and then 200 μL of the nanoparticle was inoculated into the 6-well plates. After incubation at 37° C. for four hours, the cells were collected, and total RNA was isolated from cell lysates by using a high-purity RNA isolation kit (Invitrogen). A first cDNA strand was synthesized and purified by using a PrimeScript RT kit with gDNA Eraser (Takara). qPCR was performed on a Step One real-time PCR instrument (Life Technologies) by using an SYBR Premix Ex Taq kit (Takara). Fold changes of target genes were calculated by using a 2^{-ΔΔCT} method. Experimental results are shown in FIG. 8. It can be seen from the figure that changes in the target genes CTSC mRNA, CXCR4 mRNA, and Jag1 mRNA will not be caused by the empty vector IHA/SHA/PS/KALA, indicating that the empty vector IHA/SHA/PS/KALA will not interfere with the determination of the target genes in the tumor cells.

### Example 3 Detection of Target Nucleic Acid Molecules in a Sample by Using the MB1/MB2/MVB3@KALA/PS/SHA/IHA Nanoparticle

1. Endocytosis of a YOYO-1-Hairpin@KALA/PS/SHA/IHA Nanoparticle at a Cell Line Level

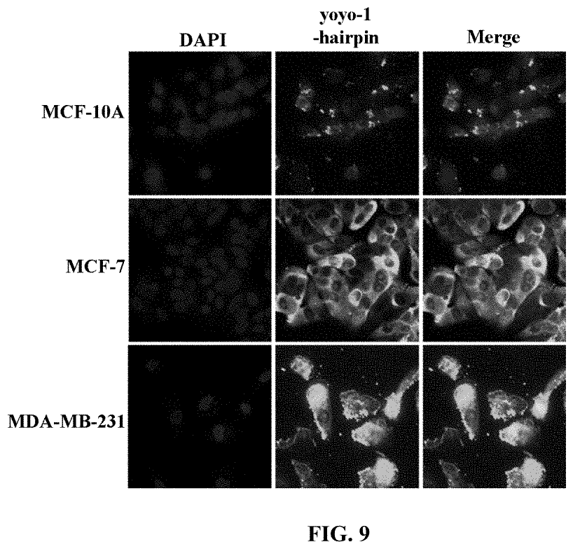

A specific implementation method is as follows. In order to eliminate the interference caused by hybridization of MB1/MB2/MB3 and mRNA targets, a YOYO-1 labeled hairpin with a random sequence (without a quenching group and a fluorescence group) is used for replacing molecular beacons, and the cell delivery ability of various nanoprobes was studied. The cellular uptake and intracellular distribution of the YOYO-1-Hairpin@KALA/PS/SHA/IHA nanoparticle were mainly determined by using a confocal laser scanning microscope (CLSM). First, tumor cells (MCF-7 refers to epithelial tumor cells, and MDA-MB-231 refers to mesenchymal tumor cells) and normal cells MCF-10A were inoculated into a 35 mm small confocal vessel for culture for 24 hours. Then 1 mL of an YOYO-1-Hairpin@KALA/PS/SHA/IHA nanoparticle solution was added for incubation with the cells for four hours. Next, the nuclei were stained with DAPI. At last, the cells were observed by using a CLSM (PerkinElmer MltraVIEW VoX). Experimental results are shown in FIG. 9. It can be seen from the figure that the cancer cells (MCF-7 and MDA-MB-231) emit stronger light than the normal cells (MCF-10A), and this is because a CD44 receptor and an EpCAM receptor or an ICAM-1 receptor are expressed on surfaces of the cancer cells. According to the results, it indicates that the synthesized YOYO-1-Hairpin@KALA/PS/SHA/IHA nanoparticle can smoothly enter the cancer cells.

2. Detection of the MB1/MB2/MB3@KALA/PS/SHA/IHA Nanoparticle at a Simulated Blood Level

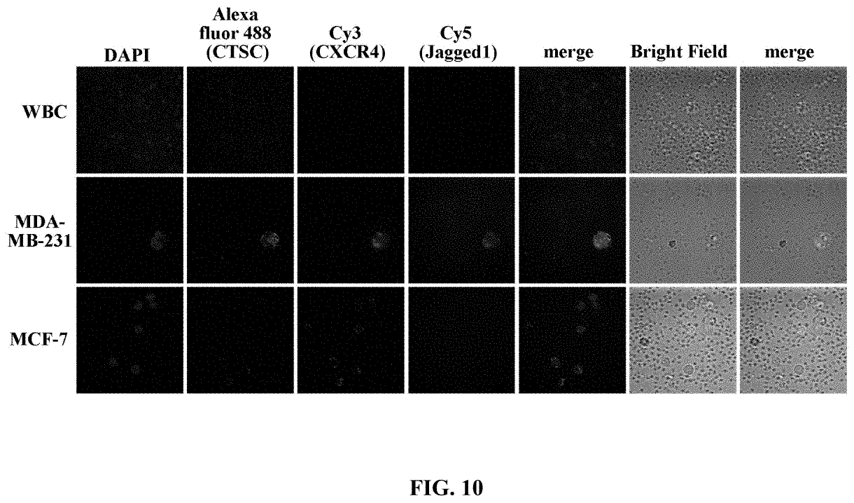

A specific implementation method is as follows. 1,000 MCF-7 and MDA-MB-231 cells were mixed in 2 ml of healthy human blood, and then subjected to standing for two hours. 1 mL of an MB1/MB2/MB3@KALA/PS/SHA/IHA nanoparticle solution was added to the blood for incubation for four hours. Then, the blood was added to a 15 ml lymphocyte separation tube, and centrifuged at a rotation speed of 800 g/min for 20 minutes. A PBMC layer was taken, and added into a small confocal vessel. The nuclei were stained with DAPI, and then observed by using a CLSM. Experimental results are shown in FIG. 10. The highly metastatic cancer cells (MDA-MB-231) emit strong fluorescence with three different colors, the lung metastasis cancer cells emit fluorescence with only one color, and red blood cells and white blood cells hardly emit fluorescence. It indicates that the MB1/MB2/MB3@KALA/PS/SHA/IHA nanoparticle can be accurately targeted to the cancer cells in a complex blood environment, and detect expression levels of CTSC mRNA, CXCR4 mRNA, and Jag1 mRNA in different metastatic cancer cells.

3. Detection of the MB1/MB2/MB3@KALA/PS/SHA/IHA Nanoparticle in Real Patient Blood

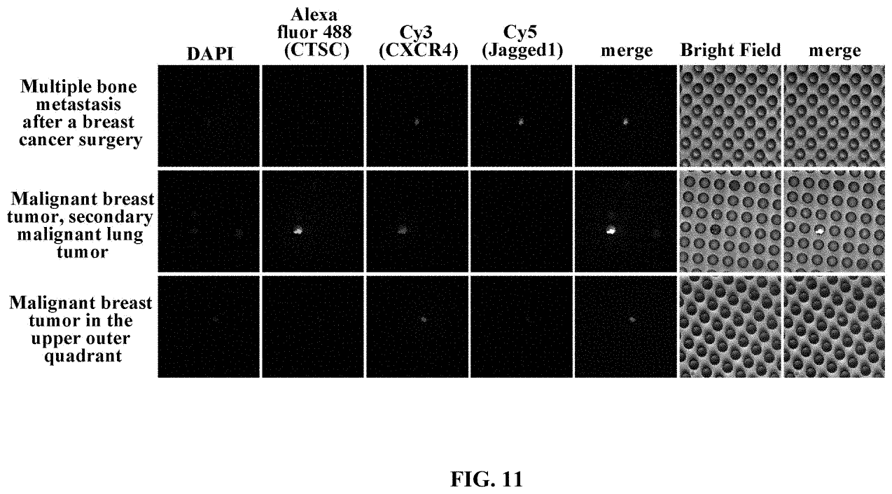

A specific implementation method is as follows. 2 ml of blood was taken from a cancer patient, and added into an EDTA anticoagulation tube. Then, 1 ml of an MB1/MB2/MB3@KALA/PS/SHA/IHA nanoparticle solution was added to the blood for incubation for four hours. The blood was put on a filtering membrane with a diameter of 70 μm for filtration, and the filtering membrane was collected in a small confocal vessel. The nuclei were stained with DAPI into blue, and then observed by using a CLSM. Experimental results are shown in FIG. 11. Each of CTCs emits purple fluorescence, indicating that the MB1/MB2/MB3@KALA/PS/SHA/IHA nanoparticle can be efficiently targeted into tumor cells in the blood of the cancer patient directly without enriching the CTCs, and also detect CXCR4 mRNA in the CTCs. In addition, breast cancer lung metastasis tumors emit green fluorescence (CTSC mRNA), and breast cancer bone metastasis tumors emit red fluorescence (Jag1 mRNA). It indicates that tumor metastasis sites can be well detected by using the method. According to the results, a favorable basis is provided for early detection of tumors, identification of metastasis sites of metastatic tumors, medication guidance, and prognosis evaluation.

### Comparative Example 1

The endocytosis of an YOYO-1-Hairpin@PS/SHA/IHA nanoparticle without the addition of a functional polypeptide is compared at a cell line level.

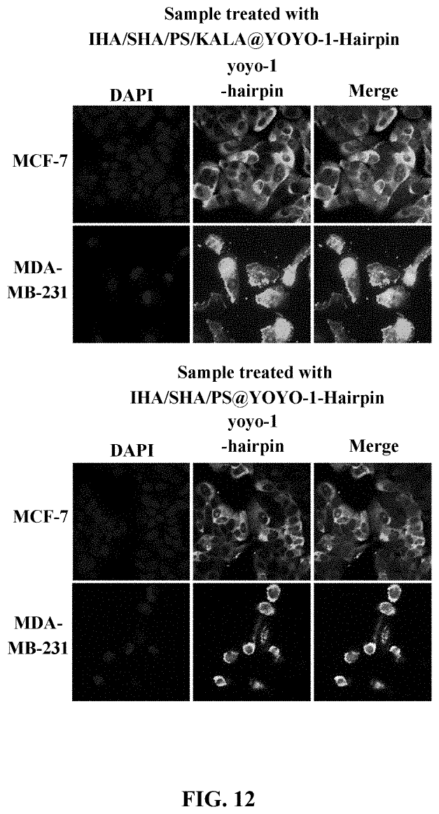

A specific implementation method is as follows. The cellular uptake and intracellular distribution of the YOYO-1-Hairpin@PS/SHA/IHA nanoparticle and the YOYO-1-Hairpin@KALA/PS/SHA/IHA nanoparticle were mainly determined by using a CLSM. First, tumor cells comprising epithelial tumor cells MCF-7 and mesenchymal tumor cells MDA-MB-231 were inoculated into a 35 mm small confocal vessel for culture for 24 hours. Then 1 mL of an YOYO-1-Hairpin@PS/SHA/IHA nanoparticle solution and 1 mL of an YOYO-1-Hairpin@KALA/PS/SHA/IHA nanoparticle solution were separately added for incubation with the cells for 4 hours. Next, the nuclei were stained with DAPI. At last, the cells were observed by using a CLSM (PerkinElmer MltraVIEW VoX). Experimental results are shown in FIG. 12. It can be seen from the figure that the YOYO-1-Hairpin@KALA/PS/SHA/IHA nanoparticle emits stronger fluorescence than the YOYO-1-Hairpin@PS/SHA/IHA nanoparticle, and this is because the cellular uptake and endosomal escape can be promoted by a functional polypeptide KALA. Therefore, the functional polypeptide KALA is introduced to the core of a nanoprobe to more efficiently improve the delivery efficiency of molecular beacons. According to the results, it indicates that compared with the YOYO-1-Hairpin@PS/SHA/IHA nanoparticle without the addition of a functional polypeptide, the synthesized YOYO-1-Hairpin@KALA/PS/SHA/IHA nanoparticle has better ability to enter cells.

The foregoing descriptions are exemplary implementations of the disclosure, and are not intended to limit the scope of the disclosure. It should be noted that, a person of ordinary skill in the art may make some improvements and variations without departing from the principle of the disclosure and the improvements and variations shall fall within the protection scope of the disclosure.

What is claimed is:

1. A molecular beacon nanoprobe for directly detecting organ-specific metastasis related genes of tumor cells in peripheral blood, wherein the molecular beacon nanoprobe is a nanoparticle formed by self-assembly of a polymer material, a positively charged protein, a functional polypeptide, and a molecular beacon of organ-specific metastasis related RNA biomarkers of tumor cells.

2. The molecular beacon nanoprobe of claim 1, wherein the polymer material comprises any one of aptamer conjugated hyaluronic acid, peptide conjugated hyaluronic acid, aptamer conjugated carboxymethyl chitosan, peptide conjugated carboxymethyl chitosan, aptamer conjugated sodium alginate, peptide conjugated sodium alginate, aptamer conjugated heparin sodium, and peptide conjugated heparin sodium.

3. The molecular beacon nanoprobe of claim 2, wherein an aptamer comprises at least one of AS1411, SYL3C, MUC1-aptamer, EGFR-aptamer CL4, and ICAM-1-aptamer; and a polypeptide comprises any one of a cell-penetrating peptide TAT, a CXCR4 targeting peptide T22, a VCAM-1 targeting peptide VHPKQ, and a fusion peptide formed by fusion of these peptides.

4. The molecular beacon nanoprobe of claim 1, wherein the positively charged protein comprises any one of protamine, histone, and lysozyme.

5. The molecular beacon nanoprobe of claim 1, wherein the molecular beacon of organ-specific metastasis related genes of tumor cells comprises at least one of a tumor marker molecular beacon, a tumor brain metastasis marker molecular beacon, a tumor lung metastasis marker molecular beacon, a tumor bone metastasis marker molecular beacon, and a tumor liver metastasis marker molecular beacon; and the tumor marker molecular beacon comprises a 5′ end labeled with a fluorescence group and a 3′ end labeled with a fluorescence quenching group.

6. The molecular beacon nanoprobe of claim 1, wherein the functional polypeptide comprises any one of a KALA peptide, other penetrating peptides, a targeting peptide, and a fusion peptide.

7. The molecular beacon nanoprobe of claim 1, wherein a nucleic acid capable of being detected by the molecular beacon nanoprobe comprises any one of miR-21, miR-221, CXCR4 mRNA, CTSC mRNA, Jagged1 mRNA, Ki67 mRNA, and EGFR mRNA.

8. A method for preparing the molecular beacon nanoprobe for directly detecting organ-specific metastasis related RNA biomarkers of tumor cells in peripheral blood of claim 1, the method comprising:
(1) adding a certain amount of the positively charged protein and the functional polypeptide to deionized water to prepare a solution A, adding a certain amount of a molecular beacon solution to deionized water to prepare a solution B, and adding the solution A dropwise to the solution B for uniform mixing; and
(2) adding the polymer material to the mixed solution obtained in step (1) for continuous and uniform mixing to obtain the molecular beacon nanoprobe.

9. The method of claim 8, wherein in (2), after the polymer material is added, a mass ratio of the positively charged protein to the functional polypeptide to the molecular beacon to the functionalized polymer material in the mixed solution is 30:(1-3):(1-2.5):(5-15), and the positively charged protein has a concentration of 1-3 μg/μL.

10. A method for directly detecting organ-specific metastasis related genes of tumor cells in peripheral blood comprising preparing a reagent for detecting tumors, tumor brain metastasis, tumor lung metastasis, tumor liver metastasis, and tumor bone metastasis using the molecular beacon nanoprobe of claim 1.
